(19) United States
(12) Patent Application Publication
(10) Pub. No.: US 2020/0375658 A1
(43) Pub. Date: Dec. 3, 2020

(54) RESPONSE MONITORING

(71) Applicants: The University of Sydney, New South Wales (AU); Western Sydney Local Health District, New South Wales (AU)

(72) Inventors: Pierre Qian, New South Wales (AU); Michael Anthony Barry, New South Wales (AU)

(73) Assignees: The University of Sydney, New South Wales (AU); Western Sydney Local Health District, New South Wales (AU)

(21) Appl. No.: 16/994,134

(22) Filed: Aug. 14, 2020

Related U.S. Application Data
(63) Continuation of application No. PCT/AU2019/050202, filed on Mar. 8, 2019.

(30) Foreign Application Priority Data
Mar. 9, 2018 (AU) 2018900779

(51) Int. Cl. A61B 18/14; A61B 18/18; A61B 90/00

(52) U.S. Cl. CPC A61B 18/1492; A61B 2018/00577; A61B 90/37; A61B 18/1815

(57) ABSTRACT

The invention concerns a method for intra-operative monitoring of the effectiveness of transcatheter renal denervation in a patient, to assist in guiding the procedure and in particular for identifying a physiological procedural endpoint. Through aorticorenal ganglia pace-capture, renal sympathetic nerve function can be assessed. In accordance with the invention, sustained reduction or abolition of renal vasoconstriction induced by the pacing is used as an indicator of successful renal denervation.

### CROSS-REFERENCES TO RELATED APPLICATIONS

This application claims priority to and is a continuation of International Patent Application No. PCT/AU2019/050202, filed Mar. 8, 2019; which claims priority from AU Patent Application No. 2018900779, filed Mar. 9, 2018. The entire contents of each of the PCT/AU2019/050202 and the AU Patent Application No. 2018900779 are hereby incorporated by reference in their entirety for all purposes.

### FIELD OF THE INVENTION

This invention relates to response monitoring. More particularly, the invention concerns a method for intra-operative monitoring of the effectiveness of renal denervation in a patient, to assist in guiding the procedure.

### BACKGROUND OF THE INVENTION

Hypertension is the most commonly diagnosed medical condition and a global health crisis, affecting approximately 1 in 3 adults and causing deaths from cardiovascular disease at a rate of 9.4 million deaths a year world-wide. Globally, hypertension has seen an alarming rise in recent times, with 600 million people affected in 1980 growing to 1 billion in 2008, with the highest prevalence rates in developing countries. For every 20 mmHg increase in systolic pressure and 10 mmHg in diastolic pressure above 115 mmHg/75 mmHg, there is a doubling of cardiovascular mortality. It is estimated that if prevention of cardiovascular disease is not addressed, the global economic toll from 2011 to 2030 will total 15.6 trillion US dollars.

In a western population, despite the availability of medical therapy, only half of patients with hypertension achieve target blood pressure control, and up to 1 in 8 have resistant hypertension, defined as uncontrolled blood pressure despite using 3 or more antihypertensives of different classes at maximal tolerated doses. Clearly, current medical therapies for hypertension, even if ubiquitously available, will be inadequate to fully remedy this growing epidemic. Without new therapies for hypertension, immense health and socioeconomic consequences will have to be faced.

The paradigm that renal nerve hyperactivity contributes to driving resistant hypertension via increasing total body sympathetic output and promoting renal salt and fluid retention is supported by numerous physiological studies and by the historical success of surgical renal denervation for treating hypertension. More recently, transcatheter radiofrequency ablation from within the renal artery has emerged as a potential method for renal denervation, supported by efficacy data from controlled trials and clinical registry data.

A microwave transcatheter ablation device and method of its use is described in International Patent Application Publication No. WO 2016/197206. This device is designed for controlled circumferential denervation in a renal artery, the device introduced via a peripheral artery such as the femoral artery, within a guiding sheath which engages the ostium of the renal artery. The entire content of WO 2016/197206 is incorporated herein by reference.

Notwithstanding the potential therapeutic benefits of renal denervation procedures, trial results have been mixed. The largest randomised controlled trial to date, Symplicity HTN-3, failed to show efficacy when the intervention was compared to a sham procedure. After radiofrequency renal denervation therapy, norepinephrine spill-over measurements in patients have revealed incomplete and non-uniform denervation and subsequent large animal studies have shown the capacity for histological neuroregeneration and physiological recovery of renal nerve function after radiofrequency ablation. Without an effective, consistent and durable method to perform transcatheter renal denervation, there are real challenges in assessing with certainty in clinical trials its potential as a therapeutic intervention.

An important cause for the inconsistent efficacy of transcatheter denervation procedures is the lack of a means to monitor the effect of catheter ablation on renal nerve activity during the procedures. This lack of an intra-operative endpoint means that it is not possible to ascertain whether the ablations performed have led to renal nerve injury and how complete this injury is.

Renal nerve stimulation is known to dramatically reduce renal blood flow through activation of efferent renal nerves and cause arterial vasoconstriction while increasing blood pressure immediately though activation of afferent sensory fibres that increase peripheral arterial resistance.

Studies of renal nerve stimulation during open surgery in animal models have been conducted in the past, and have demonstrated that renal nerve stimulation can lead to renal vasoconstriction together with a hypertensive response. As far as the present inventors are aware, concurrent efferent response of renal vasoconstriction has never been examined with the afferent response of blood pressure change, because nerve stimulation has been applied within (or very close to) the renal artery itself, thus precluding meaningful assessment of the effect of electrical stimulation on properties of the renal artery (such renal vascular calibre, renal artery flow, pressure drop or vascular resistance), due to the difficulty of segregating the effect of pacing on renal nerve stimulation from that of direct mechanical stimulation of the renal vasculature.

Furthermore, from the relevant literature, it has remained uncertain whether some blood pressure responses when pacing are due to stimulation of pain fibres in the retroperitoneal region. Hence, it seems clear that blood pressure elevation from pacing within the region of the renal arteries cannot be a basis of a reliable technique to localise and stimulate renal nerves.

In regard to the relevant prior art, direct aorticorenal ganglion (ARG) pacing in open surgery in dogs and its effect on blood pressure and heart rate has been studied. This study suggested its possible use in respect of observing the effect of local denervation. Further, the prior art includes literature publications concerning renal arterial vasodilation (in human patients and in dogs) after radiofrequency renal denervation. However, these studies required waiting between 30 minutes and 6 months after the ablation before the effect could be observed. Clearly, this not a practical method for guiding any sort of surgical procedure.

In summary, no techniques have been hitherto developed for efferent renal nerve assessment during transcatheter renal denervation procedures.

### PRIOR ART CITATIONS

‘Renal Artery Vasodilation May Be An Indicator of Successful Sympathetic Nerve Damage During Renal Denervation Procedure’; WeijieChen, Huaan Du, Jiayi Lu, Zhiyu Ling, Yi Long, YanpingXu, PeilinXiao, Laxman Gyawali, Kamsang Woo, Yuehui Yin and Bernhard Zrenner; 16 Nov. 2016, Scientific Reports 6:37218 DOI: 10.1038/srep37218. (https://www.ncbi.nlm.nih.gov/pmc/articles/PMC5110962)
‘Effects of Renal Denervation on Renal Artery Function in Humans: Preliminary Study’; Doltra A, Hartmann A, Stawowy P, Goubergrits L, Kuehne T, et al.; 22 Mar. 2016; PLOS ONE 11(3): e0150662. (https://doi.org/10.1371/journal.pone.0150662)

There is therefore a need to provide a means of reliable intraprocedural monitoring of the effect of renal artery denervation, ideally to afford a procedural endpoint for the denervation.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a person skilled in the art.

### BRIEF SUMMARY OF THE INVENTION

In a first aspect, the invention provides a method for monitoring renal denervation in a patient through transcatheter ablation, the method including: introducing one or more intraluminal electrodes via a peripheral vein and/or artery of the patient; applying an electrical pacing stimulus by way of the one or more electrodes at a particular site or sites in the vicinity of the renal artery ostium; monitoring stimulation of the renal nerves and or one or more proximate ganglia involved in kidney innervation by observing blood pressure response and/or renal artery calibre changes, an observation of resulting increased blood pressure and/or renal artery vasoconstriction indicating an appropriate site application of the electrical pacing stimulus; performing a renal denervation procedure by transcatheter ablation; monitoring the effect on renal artery calibre after or during the ablation procedure to determine efficacy of denervation.

Monitoring the effect on renal artery calibre after or during the ablation procedure may involve further observing renal artery calibre changes in response to applied electrical pacing stimulus at said particular site or sites, or observing dilation of the renal artery in response to renal denervation after sustained renal arterial vasoconstriction produced by the application of the electrical pacing stimulus prior to the denervation.

As will be understood, the invention provides an effective method of monitoring the effect of transcatheter ablation of renal nerves, so providing feedback to a surgeon at the time of the denervation, to assist in monitoring the effectiveness and in guiding the procedure, eg. the dosing and the localisation of the ablation. Hence, the invention can provide a reliable endpoint for the denervation intervention.

Further, it will be understood that the technique provides a patient-specific way of testing a ‘before and after’ response change to guide renal denervation. The applied pacing increases the state of activation of efferent renal sympathetic nerves, which during procedural sedation may allow the renal artery to be otherwise in a dilated state, so to create an increased local sympathetic tone. The relief of this sympathetic tone can indicate a reliable endpoint for the denervation procedure.

The renal artery (and its blood flow) is monitored by one or more known methods, including but not limited to:
a) By angiogram of the renal artery;
b) By bioimpedance measurement of the renal artery lumen between proximal and distal points within the artery (or from the artery to the renal vein); as the lumen decreases (or renal vascular bed contracts), the impedance will rise;
c) By thermodilution measurement of renal artery flow; as the lumen decreases, flow will also decrease;
d) By ultrasound imaging of the kidneys showing changes in Doppler blood flow either in the renal artery or the renal tissue itself.

As will be understood, other suitable techniques for monitoring the renal artery can be employed. For example, a suitable pressure-temperature sensor-tipped wire (eg. 0.014″ wire) can be inserted and used both to determine pressure and to take thermodilution measurements. Vascular resistance can be determined once the pressure gradient and the flow rate are known.

In a preferred form, the or each intraluminal electrode is provided in a catheter device introduced into the inferior vena cava and/or aorta percutaneously via a peripheral vein or artery.

Preferably, the electrical pacing stimulus is applied to a target site or sites in a region between 10 cm above and 10 cm below (preferably between 5 cm above and 5 cm below) the renal artery ostium, in order to identify a site or sites that result simultaneously in an increased blood pressure response and renal artery vasoconstriction, the response occurring within a period of 2 minutes (preferably within a period of 30 seconds) from the commencement of the application of the electrical pacing stimulus.

The target sites are small, generally of less than 10 mm diameter, and found by experiment. The inventors have determined that the target sites generally lie between the ipsilateral renal artery ostium and a point approximately 5 cm above it, closely associated with the aorta, posterior aspect of the inferior vena cava and the adipose tissue in that region. When found, pacing of the points has a nearly immediate effect on blood pressure and renal artery calibre and can be easily identified from a rise in blood pressure tracings, with or without other means of determining renal artery calibre. Preferably, both blood pressure elevation and renal arterial vasoconstriction are used to confirm capture of the ipsilateral ARG.

The electrical pacing stimulus may take the form of relatively high frequency pacing. Preferably, this is at a frequency of at least 10 Hz, and may be up to around 2 kHz. For example, the electrical impulse may be of 2 ms duration applied every 100 ms. Preferably the electrical stimulus is a current in the range of 10 mA to 30 mA. Suitable electrical pacing can be obtained from a conventional cardiac pacing console, such as the Micropace EPS320, delivered in such a fashion as to minimise muscular stimulation if encountered.

The electrical pacing stimulus may be applied as a unipolar pacing between the catheter electrode and a surface indifferent electrode, or alternatively as bipolar pacing between two intraluminal electrodes applied at appropriate sites.

Trials have indicated that appropriate sites are approximately 3-4 cm above the renal artery ostium and may be paired, one on either side of the aorta, these sites understood to correspond substantially to the ARG. In one approach, therefore, the electrical pacing is applied to the right side of the aorta by way of a catheter device introduced into the inferior vena cava, and to the left side of the aorta by way of a catheter device introduced into the aorta. Trials have also shown that it may be possible, depending on anatomical relationship, to capture both ARGs from the IVC, IVC pacing being preferable due to the lower risk associated with access via the venous system. Pacing is performed on the right and left sites at the time of denervation of the respective kidney.

The efficacy of the denervation procedure may be determined by:
(1) the return of renal artery calibre during or soon after the renal nerve ablation to pre-pacing dimensions at a site where repeated or prolonged application of the electrical pacing stimulus was observed to produce sustained renal vasoconstriction, and/or
(2) failure to observe reversible renal artery constriction with application of the electrical pacing stimulus at a site or sites where said application of the electrical pacing stimulus was previously observed to produce reversible renal vascular constriction.

The transcatheter renal ablation procedure is preferably carried out by a circumferential renal denervation system which does not create significant renal artery spasm which may give rise to vasoconstriction during operation (thus potentially interfering with real-time monitoring of renal vascular response). In one form, a transcatheter microwave ablation system is used. As will be understood, alternative ablation procedures may be employed, such as targeted spot neural ablation without arterial involvement.

As noted above, the invention addresses the need for a procedural endpoint for renal artery denervation, and in particular the need for a physiological intraoperative endpoint in transcatheter renal artery denervation. Endovascular pace-capture of aorticorenal ganglia can produce renal arterial vasomotor responses to provide operator feedback regarding efferent renal nerve function.

Further aspects of the present invention and further embodiments of the aspects described in the preceding paragraphs will become apparent from the following description, given by way of example and with reference to the accompanying figures.

### BRIEF DESCRIPTION OF THE DRAWINGS

FIG. 1 is an illustration demonstrating relief of repetitive ARG pacing induced vasoconstriction with circumferential renal artery denervation;

FIG. 2 illustrates results showing the haemodynamic and vasoconstrictive responses to the ARG pacing;

FIG. 3 illustrates the right putative ARG site injected;

FIG. 4 illustrates the left putative ARG site injected;

FIG. 5 illustrates Ganglionic tissue observed at injection labelled sites histologically; and

FIG. 6 provides a diagrammatic illustration of the process of the invention.

### DETAILED DESCRIPTION OF THE INVENTION

It will be understood that the invention disclosed and defined in this specification extends to all alternative combinations of two or more of the individual features mentioned or evident from the text or drawings. All of these different combinations constitute various alternative aspects of the invention.

As noted above, renal nerve stimulation is known to reduce renal blood flow through activation of efferent renal nerves and causing arterial vasoconstriction, while increasing blood pressure though activation of afferent sensory fibres that increase peripheral arterial resistance. With this in mind, the inventors of the present invention looked at ways to stimulate the renal nerves or a nearby ganglion innervating the kidney, with a view to the renal vascular changes providing a testable procedural endpoint during transcatheter ablation for renal denervation.

In accordance with an embodiment of the invention, using cardiac electrophysiology catheters with an end electrode, the inferior vena cava (IVC) or aorta is entered percutanously via a peripheral vein or artery. High frequency unipolar pacing at greater or equal to 10 Hz using 10 to 30 mA is performed in the vicinity of the renal artery ostium and up to 5 cm above and below to find sites that produce simultaneously an increased blood pressure response and renal artery vasoconstriction within 2 minutes of pacing. These sites tend to be around 3-4 cm above the renal artery ostium and are often paired one on either side of the aorta. The right side is generally accessible by pacing from the IVC, but the left sided structure can require pacing from within the aorta. These sites may correspond to the ARG.

Pacing is performed prior to circumferential renal denervation and the efficacy of denervation gauged by 1) the return of renal arterial calibre during and immediately after ablation to pre-pacing dimensions after renal vasoconstriction is produced by repetitive or prolonged pacing at the target site, and 2) the loss of reversible renal constriction with pacing at the target site where reversible renal vascular constriction was previously demonstrated with pacing. This method is used in conjunction with a circumferential renal denervation system which during operation does not create significant renal artery spasm and which does not occlude renal artery flow (allowing renal arterial vascular changes to be assessed), such as the system described in International Patent Application Publication No. WO 2016/197206.

Alternative pacing methods or devices can be used, such as bipolar pacing from a catheter in the IVC to another in the aorta. Alternatively, devices that have multiple electrodes that can be placed in the IVC or aorta can be used, and pacing from selected electrodes or between selected electrode pairs can increase the chance of pace-capture of this putative ARG.

### Testing and Validation

Progressive prototype developments and extensive animal testing and in vitro testing have validated the feasibility of the invention.

### A. Experiment 1

Methods:

High-frequency pacing was performed at multiple sites in the inferior vena cava (IVC) and aorta at 25 mA and 10 Hz in 8 sheep. Aorticorenal ganglia pace-capture was inferred if a hypertensive and renal vasoconstrictor response was simultaneously observed. Renal artery dimensions were measured with quantitative coronary analysis software.

Results:

Discrete regions 32±4 mm superior to the right renal artery ostium and 38±3 mm superior to the left renal artery ostium could be captured from the IVC and left anterior aorta respectively, correlating to ganglionic tissue seen histologically. Pacing produced a mean arterial pressure increase of 23 (IQR 18-28) mmHg without significant heart rate change, and ipsilateral renal artery mean diameter change of −13±11%, p=0.0005, without consistent effect on the contralateral renal artery −5±14%, p=0.18.

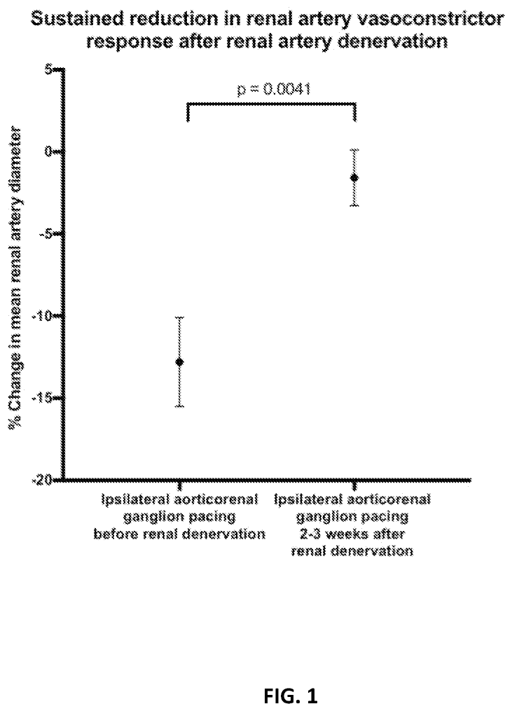

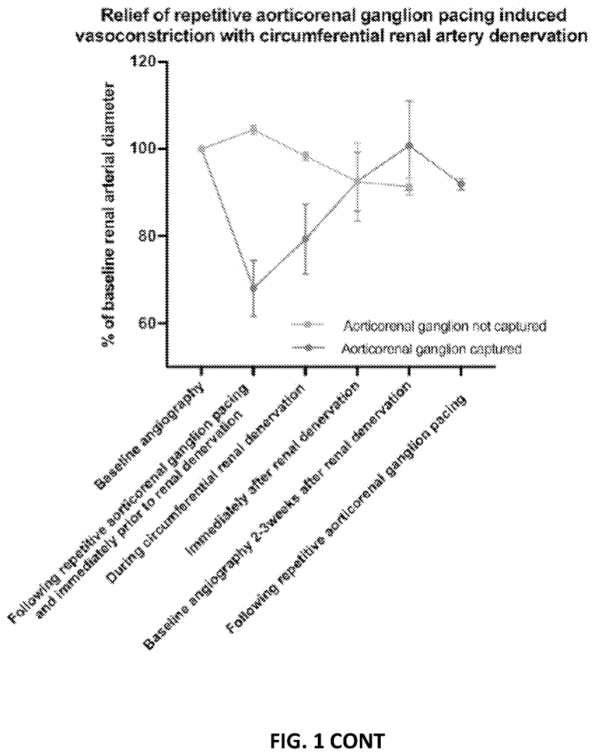

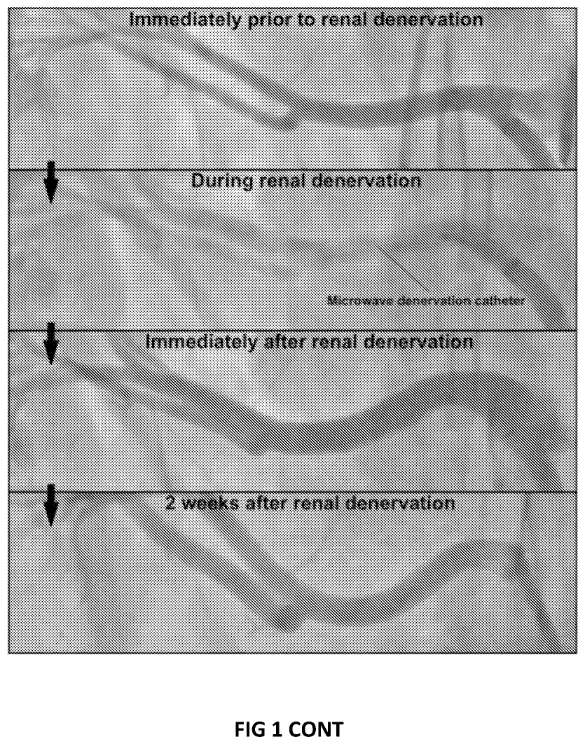

The results are illustrated in FIG. 1, demonstrating relief of repetitive ARG pacing induced vasoconstriction with circumferential renal artery denervation. The angiograms (third page of FIG. 1) show the renal artery state immediately prior to, during, immediately after and two weeks after the renal denervation. The graphs (first two sheets of FIG. 1) show renal arterial diameter at the different stages, and the sustained reduction in vasoconstrictor response after renal artery denervation.

Conclusion:

High-frequency pacing from the IVC and aorta appears feasible for localising aorticorenal ganglia that produce consistent ipsilateral renal arterial vasoconstriction and offers a potential means to test renal sympathetic efferent nerve function during transcatheter renal artery denervation.

### B. Experiment 2

Methods:

8 sheep underwent unilateral microwave renal artery denervation after attempts to identify and repetitively pace the ipsilateral ARG to maximise renal artery vasoconstriction. Capture of the ARG was inferred by concurrent hypertensive and ipsilateral renal vasoconstrictor responses during high-frequency pacing at 25 mA and 10 Hz (100 ms period, 2 ms pulse) from the inferior vena cava and the aorta.

Results:

In 6 of 8 renal arteries prior to denervation, pacing reduced renal arterial diameter from 5.8±1.2 mm to 4.0±1.5 mm, p value=0.007. Whenever vasoconstriction was induced by pacing, microwave renal denervation caused progressive vasodilation during ablation to restore renal artery diameter, 5.3±0.7 mm vs 5.8±1.2 mm at baseline, p=0.14. At 2-3 weeks, the ipsilateral aorticorenal ganglia could no longer be pace-captured in three of six arteries where it was previously possible and, in the remaining three, pacing produced insignificant changes in renal arterial diameter 5.7±0.5 mm to 4.8±1.3 mm, p=0.38. Renal cortical norephinephrine content on the denervated side was reduced by 73%, p=0.0004.

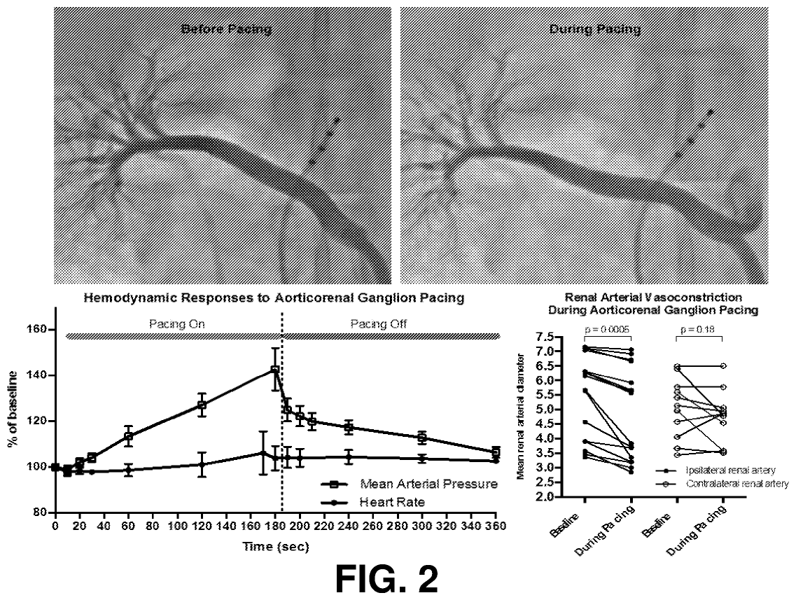

The results are illustrated in FIG. 2, showing the haemodynamic and vasoconstrictive responses to the ARG pacing.

Conclusion:

When renal sympathetic tone is increased, effective circumferential renal artery denervation may be appreciated by immediate renal artery vasodilation and diminished vasoconstrictive response to ARG pacing.

### C. Experiment 3

Methods:

In 3 sheep, using a modified radiofrequency ablation catheter with a retractable needle tip, ink mixed with intravenous contrast (50:50%) was injected under fluoroscopic guidance, at the site of pacing which elicited ipsilateral renal arterial constriction together with blood pressure elevation. Histological analysis was performed after formalin fixation and sectioning every 4 mm in the area of the retroperitoneum where the stain was evident.

Results:

4 pacing sites in the 3 sheep yielded ipsilateral renal artery constriction concurrent with hypertensive responses. Ink injection was directed into the perivascular adipose tissue posterior to the IVC and/or anterior to the aorta. Histological analysis demonstrated abundant ganglionic tissue at injection sites.

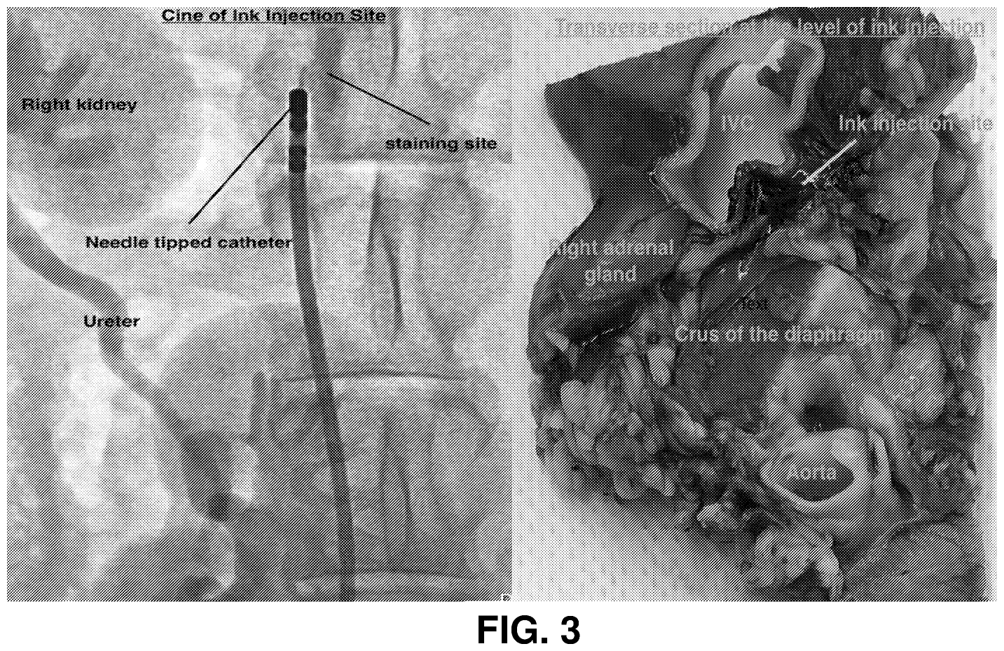

Right putative ARG site injected: FIG. 3.

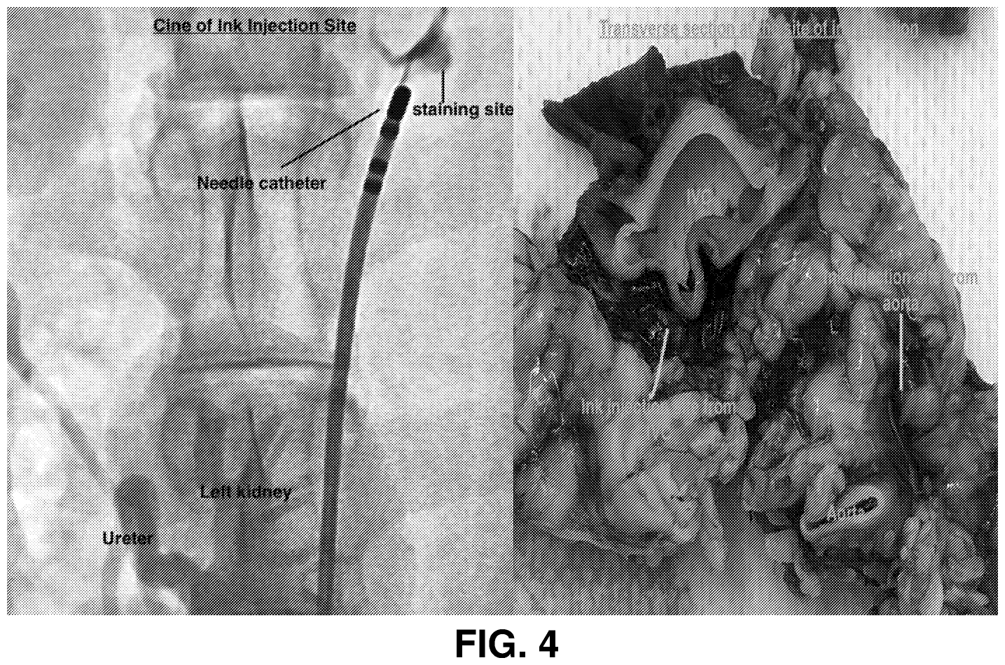

Left putative ARG site injected: FIG. 4.

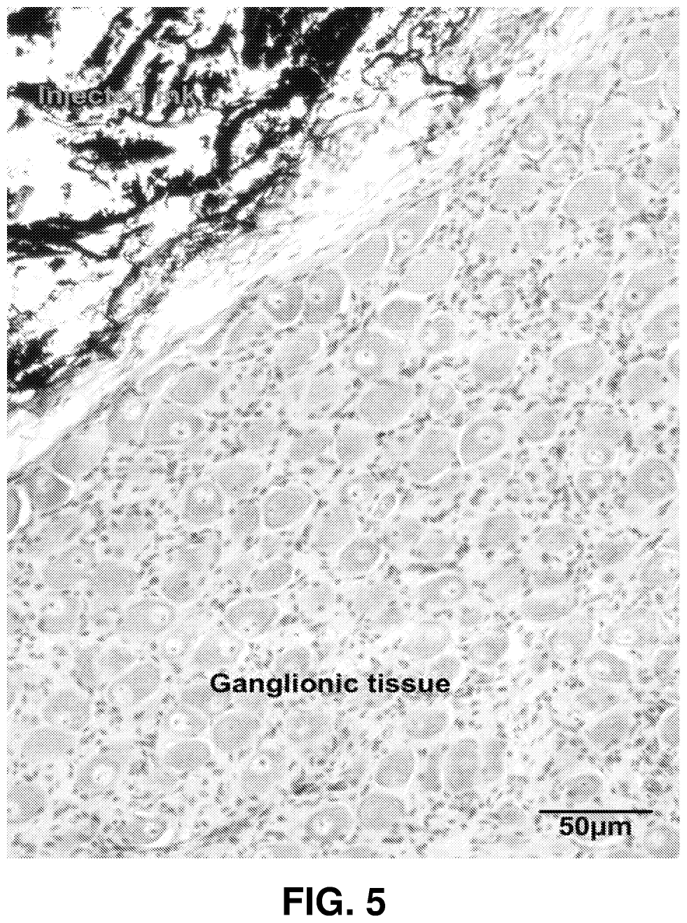

Ganglionic tissue was observed at injection labelled sites histologically: FIG. 5.

Conclusion:

sites with pacing response consistent with stimulation of ARG correlate with histological evidence of ganglionic tissue.

The results of these experiments clearly demonstrate that renal arterial vasoconstriction from high renal sympathetic tone can allow intraprocedural arterial vasodilation to serve as a renal denervation endpoint, thus assisting in guiding the dosing of renal denervation procedures (such as transcatheter circumferential renal denervation) to achieve more complete or more elective renal denervation, so improving procedural efficiency.

Moreover, the results demonstrate that pace-capture of the ARG may enable physiological testing of renal sympathetic efferent nerves.

Further tests carried out by the inventors using both RF and MW ablation provided additional confirmation of the above findings, namely that it is possible to localise ARG using transvascular pacing through observation of renovascular and haemodynamic changes, that the pacing site corresponding to a sympathetic ganglion is indeed an ARG (through demonstration of ipsilateral renal denervation with ganglion ablation), and that renal artery denervation can abolish ARG pacing-induced renal vasoconstriction. Again, histological assessment was used to confirm the correlation of the pacing sites with sympathetic ganglionic tissue.

Additional findings from these further tests (providing inter alia further evidence that the ARG was successfully pace-captured) included:
The renovascular changes were lateralised and therefore consistent with a neurogenic rather than humoral response.
Ink injection and ablation demonstrated a sympathetic ganglion was present at the pace capture site.
Ablation injury to the ganglion was associated with ipsilateral renal denervation, implicating its role in innervating the ipsilateral kidney. It was noted that the left ARG was more difficult to locate with pacing than the right, likely due to its variable depth within periaortic fat and the routine transaortic approach for the left side used in the trials. Histological analysis suggested that a paired leftward sympathetic ganglion (likely the left ARG) is often close to the ostium of the left renal vein and therefore may be accessible from the left aspect of the IVC. The vasodilatory response with microwave ablation was seen only if the ipsilateral ARG was captured, suggesting that the mechanism is likely due to relief of sympathetic tone rather than a direct effect on the vascular smooth muscle.

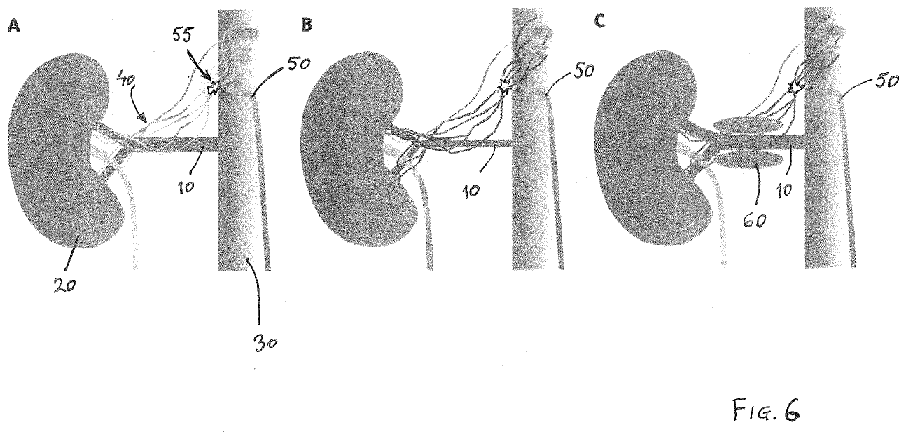

FIG. 6 provides a diagrammatic illustration of the process of the invention, showing renal artery 10 supplying blood to kidney 20, from aorta 30 (FIG. 6A). The aorticorenal ganglia and renal sympathetic fibres are indicated by reference 40. End-electrode equipped catheter 50 produces electrical pacing 55 at a suitable site, selected to correspond to a sympathetic ganglion, resulting in renal vasoconstriction (and concurrent blood pressure elevation) in artery 10, as illustrated in FIG. 6B. Transcatheter renal denervation (indicated by ablation zone 60 in FIG. 6C) blocks renal nerve activation, reducing or abolishing renovascular response to the ARG pacing.

### Further Details of Test Procedures and Equipment Used

In these tests, high frequency unipolar transvascular pacing at 10 Hz at up to 25 mA was applied using a Micropace EP stimulation source supplying either a deflectable Webster quadrapolar catheter or a 3.5 mm Thermocool ablation catheter (Biosense Webster). Renal angiography was performed using either an 8.5F epicardial Agilis Sheath (St Jude Medical) or a 6F diagnostic angiography catheter via a 7F femoral arterial short sheath. Invasive blood pressure was monitored via either a dedicated 6F short sheath inserted on the left femoral artery or from the angiography guide catheter and recorded on a Prucka CardioLab system (GE Healthcare). The tip of the pacing catheter was positioned at multiple sites above and below the level of the ipsilateral renal artery ostium. Skeletal muscle stimulation was avoided by reducing pacing current output. If no change in blood pressure was observed within 30 s of stimulation of a site, the pacing catheter tip position was moved a few millimetres to a new position.

Hemodynamic pressure data was extracted from the Prucka CardioLab system, and with main renal artery calibre determined using quantitative coronary analysis software (Siemens AG), while quantitative analysis of renal arterial tree vasoconstriction beyond the branch renal arteries was performed by (1) obtaining a digital subtraction angiography (Horos2k, version 2.0.2), (2) reducing background noise in ImageJ (ImageJ, version 1.51s) using the ‘subtract background’ function, (3) selecting a circular region of interest with a diameter defined by the first renal artery bifurcation and the furthermost point on the renal cortex, (4) obtaining a mean measure of greyscale, and (5) computing a pixel density index being the complement of greyscale (pixel density index=255-greyscale value). GraphPad Prism 7 (GraphPad Software Inc.) was used for statistical analysis.

ARG pace capture was inferred when a rise in mean invasive blood pressure within 30 s of pacing was accompanied by constriction in the ipsilateral main renal artery. After cessation of pacing, blood pressure was permitted to return to steady state, defined as less than 5 mmHg change in mean arterial pressure over 60 s. Ipsilateral and contralateral renal angiography was performed at baseline prior to pacing and at the peak of blood pressure elevation during pacing stimulation.

The invention thus provides a repeatable physiological patient-specific method to test a ‘before and after’ response change to guide renal denervation. The state of activation of efferent renal sympathetic nerves, which during procedural sedation may allow the renal artery to be otherwise in a dilated state, can be increased using pacing to create an increased local sympathetic tone, and the relief of this sympathetic tone can become a reliable endpoint for the denervation procedure.

Further, the method of locating perivascular ganglia in the manner described above also has potential future application in locating sites to apply ablation energy to produce denervation of the organ innervated by the ganglia. Such applications include renal denervation, as well as other sites in the aorta and IVC external to the renal artery.

It will be understood that the invention disclosed and defined in this specification extends to all alternative combinations of two or more of the individual features mentioned or evident from the text or drawings. All of these different combinations constitute various alternative aspects of the invention.

As used herein, except where the context requires otherwise, the term “comprise” and variations of the term, such as “comprising”, “comprises” and “comprised”, are not intended to exclude further additives, components, integers or steps.

What is claimed is:

1. A method for monitoring renal denervation in a patient through transcatheter ablation, the method including:
introducing one or more intraluminal electrodes via a peripheral vein and/or artery of the patient;
applying an electrical pacing stimulus by way of the one or more electrodes at a particular site or sites in the vicinity of the renal artery ostium;
monitoring stimulation of the renal nerves and or one or more proximate ganglia involved in kidney innervation by observing blood pressure response and/or renal artery calibre changes, an observation of resulting increased blood pressure and/or renal artery vasoconstriction indicating an appropriate site application of the electrical pacing stimulus;
performing a renal denervation procedure by transcatheter ablation;
monitoring the effect on renal artery calibre after or during the ablation procedure to determine efficacy of denervation.

2. The method of claim 1 wherein monitoring the effect on renal artery calibre after or during the ablation procedure involves:
further observing renal artery calibre changes in response to applied electrical pacing stimulus at said particular site or sites; or
observing dilation of the renal artery in response to renal denervation after sustained renal arterial vasoconstriction produced by the application of the electrical pacing stimulus prior to the denervation.

3. The method of claim 1 wherein monitoring the effect on renal artery is carried out by one or more of the following:
a) by angiogram;
b) by bioimpedance measurement;
c) by thermodilution measurement of blood flow; or
d) by ultrasound imaging.

4. The method of claim 1 wherein the or each intraluminal electrode is provided in a catheter device introduced into the inferior vena cava and/or aorta percutaneously via a peripheral vein or artery.

5. The method of claim 1 wherein the electrical pacing stimulus is applied as a unipolar pacing between the catheter electrode and a surface indifferent electrode.

6. The method of claim 1, wherein the electrical pacing stimulus is applied as bipolar pacing between two intraluminal electrodes applied at appropriate sites.

7. The method of a claim 1 wherein the electrical pacing stimulus is applied to the right side of the aorta by way of a catheter device introduced into the inferior vena cava, and to the left side of the aorta by way of a catheter device introduced into the aorta.

8. The method of claim 1 wherein the transcatheter renal ablation procedure is carried out by a circumferential microwave denervation system.
